(19) United States
(12) Patent Application Publication
(10) Pub. No.: US 2023/0233445 A1
(43) Pub. Date: Jul. 27, 2023

(54) STABLE ANHYDROUS CLEANSER CONCENTRATE FORMULATION AND METHOD OF MAKING THE SAME

(71) Applicant: One Home Brands, Inc., New York, NY (US)

(72) Inventor: Syed Humza NAQVI, Walnut Creek, CA (US)

(21) Appl. No.: 17/860,658

(22) Filed: Jul. 8, 2022

Related U.S. Application Data
(63) Continuation-in-part of application No. 17/586,424, filed on Jan. 27, 2022.

(51) Int. Cl. A61K 8/97; A61Q 19/10; A61K 8/73; A61K 8/368; A61K 8/36; A61K 8/365; A61K 8/44; A61K 8/46; A61K 8/20; A61K 8/02

(52) U.S. Cl. CPC A61K 8/97; A61Q 19/10; A61K 8/737; A61K 8/368; A61K 8/361; A61K 8/365; A61K 8/732; A61K 8/44; A61K 8/466; A61K 8/20; A61K 8/022

(57) ABSTRACT

The invention relates to stable, anhydrous concentrated cleanser formulations.

### BACKGROUND

The majority of household cleaning products on the market are in liquid or gel forms and packaged in a plastic tube, bottle, spray bottle, or pump dispenser. The problem is the packaging. Single use plastic is everywhere and it is wreaking havoc on the environment. Only 9% of all plastic is actually recycled, and packaging generates the largest portion of municipal waste (˜30%). Packaged products are inefficient for businesses and the people who buy them.

Removing the water from our formulations removes the need for single use plastic packaging and the waste that comes with it, such as packaging waste, product waste, and the waste of resources used to ship water.

Thus, a need exists for new stable formulations of cleansers in solid form that meet the needs of consumers, while also reducing the amount of waste generated in their production and shipping.

### SUMMARY OF THE INVENTION

The invention relates to stable, anhydrous cleanser concentrate formulation. The stable anhydrous cleanser concentrate formulation comprises an acidic cleaner, a pH control agent, one or more gelling agents, a drying agent, a water softener, and one or more surfactants. The formulation may further comprise at least one natural and/or synthetic fragrance. The formulation may further comprise one or more of a dye, fragrance, coloring agent, binding agent, and gelling agent. The formulation may be in powder form. The cleanser formulation may be used in personal hygiene, e.g., face wash, shampoo, body wash etc.

### DETAILED DESCRIPTION OF THE INVENTION

This disclosure relates to a stable, anhydrous, cleanser concentrate formulation. The inventors have discovered a formulation that is both good for the environment and effective for cleaning. The advantages of this anhydrous formulation over the traditional liquid cleansers include chemical stability, reduced packaging, and convenience for the consumer.

Specifically, the cleanser concentrate formulation of this disclosure includes an acidic cleaner, a pH control agent, one or more gelling agents, a drying agent, a water softener, and one or more surfactants. The formulation may further comprise at least one natural and/or synthetic fragrance. The formulation may further comprise a dye, fragrance, or coloring agent. The formulation may be in powder form. The cleanser formulation may be used in personal hygiene, e.g., face wash, shampoo, body wash etc.

As used herein, “anhydrous” refers to a water content of less than 5% by weight, e.g., less than 4%, less than 3%, less than 2%, or less than 1% by weight.

The amount of acidic cleaner in the formulation may range from about 1% to about 40%, e.g., between about 5% and about 40%, between about 10% and about 40%, between about 15% and about 40%, between about 20% and about 40%, between about 25% and about 40%, between about 30% and about 40%, between about 35% and about 40%, between about 1% and about 5%, between about 1% and about 10%, between about 1% and about 15%, between about 1% and about 20%, between about 1% and about 25%, between about 1% and about 30%, between about 1% and about 35%, between about 1% and about 40%, or between about 15% and about 35%, based on the weight of the formulation. The amount of acidic cleaner in the formulation may be about 1%, about 5%, about 10%, about 15%, about 20%, about 25%, about 30%, or about 40% by weight. Compatible acidic cleaners include inorganic acids, mineral acids, and organic acids. Exemplary organic acids include aliphatic and aromatic carboxylic acids (e.g., cinnamic acid), diacids (e.g., maleic acid), and triacids (e.g., citric acid). Preferably the acidic cleaner is a biodegradable acid. In a preferred embodiment, the acidic cleaner comprises citric acid.

The formulation may contain a pH control agent in an amount sufficient to adjust the pH when dissolved in water from about 2.0 to about 12.5. The pH of the dissolved formulation in 1 L of water may be about 2.0, about 2.5, about 3.0, about 3.5, about 4.0, about 4.5, about 5.0, about 5.5, about 2.0 to about 5.5, about 7.5, about 8.0, about 8.5, about 9.0, about 10.0, about 10.5, about 11.0, about 11.5, about 12.0, about 12.5, or about 7.5 to about 12.5. The pH control agent may range from about 1% to about 40% of the formulation, e.g., between about 5% and about 40%, between about 10% and about 40%, between about 15% and about 40%, between about 20% and about 40%, between about 25% and about 40%, between about 30% and about 40%, between about 35% and about 40%, between about 1% and about 5%, between about 1% and about 10%, between about 1% and about 15%, between about 1% and about 20%, between about 1% and about 25%, between about 1% and about 30%, between about 1% and about 35%, between about 1% and about 40%, or between about 15% and about 35%, based on the weight of the formulation. The amount of acidic cleaner in the formulation may be about 1%, about 5%, about 10%, about 15%, about 20%, about 25%, about 30%, or about 40% by weightThe pH control agent may be any agent sufficient to raise or lower the pH of the water when the formulation is dissolved in water, e.g., acids (e.g., Bronsted-Lowry acids, Lewis acids) and bases (e.g., Bronsted-Lowrybases and Lewis bases). Compatible acids include inorganic acids, mineral acids, and organic acids. Exemplary organic acids include aliphatic and aromatic carboxylic acids (e.g., cinnamic acid), diacids (e.g., malic acid), and triacids (e.g., citric acid). Preferably the pH control agent is biodegradable. Exemplary acidic pH control agents include citric acid and malic acid. Exemplary basic pH control agents include sodium carbonate and sodium bicarbonate.

The amount of chelating agent in the formulation may range from about 0.01% to about 95% by weight, e.g., between about 5% and about 95%, between about 10% and about 95%, between about 15% and about 95%, between about 20% and about 95%, between about 25% and about 95%, between about 30% and about 95%, between about 35% and about 95%, between about 40% and about 95%, between about 45% and about 95%, between about 50% and about 95%, between about 55% and about 95%, between about 60% and about 95%, between about 65% and about 95%, between about 70% and about 95%, between about 75% and about 95%, between about 80% and about 95%, between about 85% and about 95%, between about 90% and about 95%, between about 1% and about 5%, between about 1% and about 10%, between about 1% and about 15%, between about 1% and about 20%, between about 1% and about 25%, between about 1% and about 30%, between about 1% and about 35%, between about 1% and about 40%, between about 1% and about 45%, between about 1% and about 50%, between about 1% and about 55%, between about 1% and about 60%, between about 1% and about 65%, between about 1% and about 70%, between about 1% and about 75%, between about 1% and about 80%, between about 1% and 85%, between about 1% and 90%, between about 5% and about 80%, between about 10% and about 75%, between about 15% and about 70%, between about 20% and about 65%, between about 25% and about 60%, between about 30% and about 55%, between about 35% and about 50%, between about 40% and about 45%, between about 15% and about 35%, between about 25% and about 45%, between about 35% and about 55%, between about 45% and about 65%, between about 55% and about 75%, between about 65% and about 85%, or between 75% and 95% by weight. The amount of chelating agent may be about 1%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% by weight. The cleaning formulation may contain one or more chelating agents, used herein to mean a molecule or ion capable of forming multiple ionic or coordinating bonds. Chelating agents used in this disclosure may be those known in the art and include those with multiple carboxylic acid groups and salts thereof, e.g., two carboxylic acid groups (e.g., L-glutamic acid, N,N-diacetic acid, iminodisuccinic acid), three carboxylic acid groups (e.g., methylglycine diacetic acid, trisodium citrate) four carboxylic acid groups (e.g., ethylenediaminedisuccinic acid, ethylenediaminetetraacetic acid) etc.

The amount of solvent in the formulation may range from about 0.001% to about 50% by weight. The amount of solvent may be about 1%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 2% to about 10.0%, about 3% to about 7%, about 1% to about 10%, or about 4% to about 8% by weight. The cleaning formulation may contain one or more solvents selected from the group consisting of polyethylene glycol (e.g., polyethylene glycol 8000). Other glycol ethers solvents may be used, including but not limited to glycerol (i.e., glycerine or glycerin), propylene glycol, dipropylene glycol, polypropylene glycol (e.g., PPG-9), polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The solvents may provide skin moisturizing benefits.

The amount of binding agent in the formulation may range from about 1% to about 20% by weight. The amount of binding agent is suitable to form a solid and may be about 1%, about 2%, about 3%, about 4%, about 5%, about 6%, about 7%, about 8%, about 9%, about 10%, about 12%, about 15%, about 18%, about 20%, about 3% to about 7%, about 4% to about 8%, about 5% to about 10%, or about 10% to about 20%. The binding agents may be natural binders (e.g., disaccharides, polysaccharides, starches, pregelatinized starch, sodium alginate, gelatin), synthetic or semisynthetic (e.g., microcrystalline cellulose (MCC), hydroxyl propyl cellulose (HPC), polyvinyl pyrrolidone (PVP), methylcellulose, hydroxy propyl methyl cellulose (HPMC), polymethacrylates, sodium carboxy methyl cellulose, polyethylene glycol (PEG), methyl cellulose).

The amount of gelling agent in the formulation may range from about 1% to about 20% by weight. The amount of gelling agent is suitable to form a gel when the formulation is dissolved in water and may be about 1%, about 2%, about 3%, about 4%, about 5%, about 6%, about 7%, about 8%, about 9%, about 10%, about 12%, about 15%, about 18%, about 20%, about 25%, about 30%, about 35%, about 3% to about 7%, about 4% to about 8%, about 5% to about 10%, about 10% to about 15%, about 10% to about 20%, about 20% to about 30%, or about 30% to about 35%. The gelling agents may be different types of gums (e.g., acacia senegal gum, xanthan gum, guar gum, sclerotium, pectin, karaya, arabic, agar, carrageenan, alginate and combinations thereof, etc.), polymers (e.g., acrylic polymer) and mixtures thereof. Certain salts may also lead or improve gelling, including but not limited to sodium chloride, and potassium chloride. Certain cellulosics may also lead or improve gelling, including but not limited to hydroxypropyl cellulose, hydroxypropyl methylcellulose, ethylcellulose, sodium carboxy methylcellulose (cellulose gum/carboxymethyl cellulose) and cellulose (e.g. cellulose microfibrils, cellulose nanocrystals or microcrystalline cellulose).

The amount of surfactant in the formulation may range from about 10% to about 90% by weight, between about 10% and about 90%, between about 15% and about 90%, between about 20% and about 90%, between about 25% and about 90%, between about 30% and about 90%, between about 35% and about 90%, between about 40% and about 90%, between about 45% and about 90%, between about 50% and about 90%, between about 55% and about 90%, between about 60% and about 90%, between about 65% and about 90%, between about 70% and about 90%, between about 75% and about 90%, between about 80% and about 90%, between about 85% and about 90%, between about 10% and about 15%, between about 10% and about 20%, between about 10% and about 25%, between about 10% and about 30%, between about 10% and about 35%, between about 10% and about 40%, between about 10% and about 45%, between about 10% and about 50%, between about 10% and about 55%, between about 10% and about 60%, between about 10% and about 65%, between about 10% and about 70%, between about 10% and about 75%, between about 10% and about 80%, between about 10% and about 85%, between about 10% and about 80%, between about 15% and about 70%, between about 20% and about 65%, between about 25% and about 60%, between about 30% and about 55%, between about 35% and about 50%, between about 40% and about 45%, between about 15% and about 35%, between about 25% and about 45%, between about 35% and about 55%, between about 45% and about 65%, between about 50% and about 60%, between about 55% and about 75%, between about 65% and about 85%, or between about 75% and about 90% by weight. The amount of surfactant may be about 25%, about 30%, about 35%, about 40%, about 45%, about 50%, about 55%, about 60%, about 65%, about 70%, about 75%, or about 80% by weight. The surfactant may be a natural or synthetic surfactant, including anionic surfactants (e.g., sulfates: ammonium lauryl sulfate, sodium laureth sulfate, sodium myreth sulfate, sodium pareth sulfate etc.; acids: sodium lauroyl sarcosinate, sodium stearate etc.), nonionic (e.g., polyethylene glycols, sorbitans, sorbates), amphoteric, zwitterioinic, or cationic surfactant), aromatic sulfonates (e.g., alkyl benzene sulfonate). Surfactants may be added to the composition as flakes or granules to aid in dissolution rates of the composition.

The cleanser formulation may contain biologic cleaners, such as enzymes (e.g., protease, amylase, lipase, cellulose, pectinase, mannanase, and the like) and probiotics (e.g., lactobacillus, bifidobacterial, and the like). Biological cleaners may be present in an amount of about 0.01% to about 50% by weight, e.g., about 1% to about 50% by weight, between about 5% and about 50%, between about 10% and about 50%, between about 15% and about 50%, between about 20% and about 50%, between about 25% and about 50%, between about 30% and about 50%, between about 35% and about 50%, between about 40% and about 50%, between about 45% and about 50%, between about 1% and about 5%, between about 1% and about 10%, between about 1% and about 15%, between about 1% and about 20%, between about 1% and about 25%, between about 1% and about 30%, between about 1% and about 35%, between about 1% and about 40%, between about 1% and about 45%, between about 1% and about 50%, between about 5% and about 25%, between about 10% and about 30%, between about 15% and about 35%, between about 20% and about 40%, between about 25% and about 45%, between about 30% and about 50%, by weight. The amount of biological cleaners may be about 0.01%, about 0.5%, about 1%, about 2%, about 3%, about 4%, about 5%, about 10%, about 15%, about 20%, about 25%, about 30%, about 35%, about 40%, about 45%, or about 50% by weight.

The cleanser formulation may contain one or more fragrances, such as natural fragrances (e.g., essential oils) and/or synthetic fragrances and perfumes in the form of oils, crystals, powders, granules, and encapsulations. The cleanser formulation may comprise between about 0.01% and 10% fragrance by weight, e.g., between about 0.01% and about 0.05%, between about 0.05% and about 0.1%, between about 0.1% and about 0.5%, between about 0.5% and about 1%, between about 1% and about 1.5%, between about 1% and about 3%, between about 1% and about 5%, between about 1.5% and about 2.0%, between about 2.0% and about 2.5%, between about 2.5% and about 3.0%, between about 3.5% and about 4.0%, between about 4.0% and about 4.5%, between about 4.5% and about 5.0%, between about 5.0% and about 6.0%, between about 6.0% and about 7.0%, between about 7.0% and about 8.0%, between about 8.0% and about 9.0%, between about 9.0% and about 10.0%. The amount of fragrance may be about 0.01%, about 0.1%, about 0.5%, about 1.0%, about 2.0%, about 3.0%, about 4.0%, about 5.0%, about 6.0%, about 7.0%, about 8.0%, about 9.0%, or about 10.0%.

The cleanser formulation may include a preservative. Preservative in the context of this disclosure includes what is known in the art to suppress the growth of bacteria and/or fungi. The amount of the preservative in the cleanser formulation may range from about 5% to about 40%, from 5% to about 30%, from about 10% to about 30%, from about 10% to about 25%, or from about 10% to about 20%, by weight, based on the weight of the formulation. The amount of the preservative may be about 5%, about 7%, about 9%, about 11%, about 13%, about 15%,about 17%, about 19%, about 21%, about 23%, about 25%, about 27%, about 29%, about 31%, about 33%, about 35%, about 37%, about 39%, or about 40% by weight. The preservative may be sodium benzoate, potassium sorbate, gluconolactone, biocideal preservatives, and/or MIT/BIT. In some examples, sodium benzoate may also work as process aid for processing the powder to make the formulation into tablets. In some example preservatives may also provide benefits to the skin, such as anti-oxidants (e.g., vitamins E & A).

The amount of the preservative booster in the cleanser formulation may range from about 0.1% to about 15%, from about 0.5% to about 10%, from about 1% to about 10%, or from about 1% to about 5%, by weight, based on the weight of the formulation. The amount of the preservative booster may be about 0.1%, about 0.2%, about 0.3%, about 0.4%, about 0.5%, about 1%, about 2%, about 3%, about 4%, about 5%, about 6%, about 7%, about 8%, about 9%, or about 10%. Preservative boosters compatible with the present disclosure include those known in the art to aid in the suppression of bacterial and/or fungal growth by preservatives. Preservative boosters include, but are not limited to, sodium benzoate, potassium sorbate, sorbitan caprylate, and gluconolactone. In a preferred embodiment, the preservative booster is a sorbate such as potassium sorbate.

The formulation may further comprise a water softening agent. The amount of the water softener may range from 0 to 40%, e.g., from 1% to 35%, from 15% to 25%, from 5% to 15%, from 25% to 35%, from 5% to 30%, or from 10% to 25% by weight based on the weight of the dish soap. Exemplary water softener includes citric acid or a salt thereof (such as sodium citrate), ethylenediaminetetraacetic acid (EDTA), sodium gluconate, methylglycinediacetic acid trisodium salt, and tetrasodium Glutamate Diacetate.

The cleanser formulation may contain one or more dyes or coloring agents, such as Food, Drug and Cosmetic (FD&C) approved dyes and colorants. The cleanser formulation may comprise between about 0.01% and 5% dye or coloring agent by weight, e.g., between about 0.01% and about 0.05%, between about 0.05% and about 0.1%, between about 0.1% and about 0.5%, between about 0.5% and about 1%, between about 1% and about 1.5%, between about 1.5% and about 2.0%, between about 2.0% and about 2.5%, between about 2.5% and about 3.0%, between about 3.5% and about 4.0%, between about 4.0% and about 4.5%, or between about 4.5% and about 5.0%.

The cleanser formulation may contain one or more drying agents. The cleanser formulation may comprise between about 0.01% and 5% drying agent by weight, e.g., between about 0.01% and about 0.05%, between about 0.05% and about 0.1%, between about 0.1% and about 0.5%, between about 0.5% and about 1%, between about 1% and about 1.5%, between about 1.5% and about 2.0%, between about 2.0% and about 2.5%, between about 2.5% and about 3.0%, between about 3.5% and about 4.0%, between about 4.0% and about 4.5%, or between about 4.5% and about 5.0%. Dying agent known in the art to be compatible with the formulation of solid formulations (e.g., tablets and powders) may be use with the present technology. Exemplary drying agents include, but are not limited to, sodium silicate, calcium silicate, mica, kaolin, starches.

The amount of the preservative booster in the cleanser formulation may range from about 0.1% to about 15%, from about 0.5% to about 10%, from about 1% to about 10%, or from about 1% to about 5%, by weight, based on the weight of the formulation. The amount of the preservative booster may be about 0.1%, about 0.2%, about 0.3%, about 0.4%, about 0.5%, about 1%, about 2%, about 3%, about 4%, about 5%, about 6%, about 7%, about 8%, about 9%, or about 10%. Preservative boosters compatible with the present disclosure include those known in the art to aid in the suppression of bacterial and/or fungal growth by preservatives. Preservative boosters include, but are not limited to, sodium benzoate, potassium sorbate, sorbitan caprylate, and gluconolactone. In a preferred embodiment, the preservative booster is a sorbate such as potassium sorbate.

The cleanser formulation may further comprise one or more additives selected from the group consisting of oils, vitamins, flours (e.g., oat flour), emollients, and exfoliants. Each additive is between 0.1% and 10% by weight of the formulation, e.g., between 0.1% and 0.5%, between 0.5% and 1.0%, between 1.0% and 1.5%, between 1.5% and 2.0%, between 2.0% and 2.5%, between 2.5% and 3.0%, between 3.0% and 3.5%, between 3.5% and 4.0%, between 4.0% and 4.5%, between 4.5% and 5.0%, between 5.0% and 10%, between 5.0% and 5.5%, between 5.5% and 6.0%, between 6.0% and 6.5%, between 6.5% and 7.0%, between 7.0% and 7.5%, between 7.0% and 10%, between 7.5% and 8.0%, between 8.0% and 8.5%, between 8.5% and 9.0%, between 9.0% and 9.5%, or between 9.5% and 10% by weight of the formulation. In some example additives may provide benefits to the skin, such as anti-oxidants (e.g., vitamins E & A).

The stable anhydrous cleanser concentrate formulation can be formulated, among other things, as a body wash, a face wash, or a shampoo. The cleanser formulation may include an acidic cleaner, a pH control agent, one or more gelling agents, a drying agent, a water softener, and one or more surfactants. The cleanser formulation may produce a low pH solution ranging from about 2.0 to about 5.5 when dissolved in water. Alternatively, the cleanser formulation may produce a high pH solution ranging from about 7.5 to 12.5 when dissolved in water. In one embodiment, the cleanser formulation includes a surfactant, gelling agents (acacia senegal gum and xanthan gum), oat flour, preservative boosters (sodium benzoate and potassium benzoate), citric acid (acting as pH adjuster and acidic cleaner), fragrance, liquid surfactant, additives and drying agent.

The stable anhydrous cleanser concentrate formulation can be formulated as a face wash. The cleanser formulation may include an acidic cleaner, a pH control agent, one or more gelling agents, a drying agent, a water softener, and one or more surfactants. The cleanser formulation may produce a low pH solution ranging from about 4.0 to about 6.5 when dissolved in water. Alternatively, the cleanser formulation may produce a high pH solution ranging from about 7.5 to 11.0 when dissolved in water. In one embodiment, the multi-surface formulation comprises citric acid, sodium carbonate, one or more ethoxylated alcohols, methylglycineadiacetic acid, polyethylene glycol, a preservative, silicon dioxide, and magnesium stearate. The cleanser formulation may also include a fragrance and/or coloring agent.

The stable anhydrous cleanser concentrate formulation can be formulated as a shampoo. The cleanser formulation may include an acidic cleaner, a pH control agent, one or more gelling agents, a drying agent, a water softener, and one or more surfactants. The cleanser formulation may have a pH of about 5.0 to about 6.0 when dissolved in water. In one embodiment, the cleanser formulation includes citric acid, sodium carbonate, sodium lauryl sulfate, methylglycineadiacetic acid, polyethylene glycol, a preservative, and 2.2-dimethyl-1,3-dioxylane-4-methanol. The cleanser formulation may also include a coloring agent.

The stable anhydrous cleanser concentrate may also be formulated as a body wash in the form of a sheet.

### Methods for Preparing A Stable Anhydrous Cleanser Formulation

The stable anhydrous cleanser formulation can be prepared using any suitable method. It is possible to prepare the formulation in a batching process, such as in a powder form. The formulation may also be granulated in order to minimize dusting.

### Formats

The stable anhydrous cleanser concentrate formulations may be formatted as tablets, powders, or granules. The stable anhydrous cleanser concentrate formulations may also be formatted in single-use sheets.

The cleanser formulations described herein may be designed to be rinsed off, wiped off, or left on for maximum cleaning efficiency.

When the formulation is in tablet form, the tablet may range in size from about 200 mg to about 5000 mg. The tablets may be about 200 mg, about 300 mg, about 400 mg, about 500 mg, about 600 mg, about 700 mg, about 800 mg, about 900 mg, or about 1000 mg. In a preferred embodiment, the tablets are round, however other geometric shapes are contemplated.

### Methods of Using A Stable Anhydrous Cleanser Formulation

In one aspect, the invention includes a method of using any of the formulations described herein including the steps of (1) filling a bottle with water, (2) adding a cleanser formulation to the water-filled bottle, and (3) dissolving the formulation in the water by stirring or shaking.

The ratio of water to cleanser formulation described above is between 5:1 to 20:1, e.g., between 5:1 and 6:1, between 6:1 and 7:1, between 7:1 and 8:1, between 8:1 and 9:1, between 9:1 and 10:1, between 10:1 and 12:1, between 12:1 and 14:1, between 14:1 and 16:1, between 16:1 and 18:1, or between 18:1 and 20:1.

After adding the cleanser formulation to the water-filled bottle, the formulation dissolves rapidly with minimal effort. For example, the formulation may dissolve within approximately 10-15 seconds of shaking.

Each cleanser formulation, when exposed to water and stirred or shaken, will dissolve into a liquid cleansing gel. Upon experiencing dissolution of the cleanser formulation, the user may proceed with cleaning or washing as usual.

The cleanser formulation may be stored in any suitable container, such as but not limited to plastic, glass, aluminum, ceramic, or acrylic container. The container may contain a desiccant. The container may be re-usable and refilled with new formulation as needed.

### EXEMPLIFICATION

Materials used in the following Examples and their sources are listed below.

### Example 1

| Function | % Weight | Identity |
|---|---|---|
| Surfactant | 50-60% | |
| Gelling Agent | 10-15% | Acacia Senegal Gum/ |
| | | Xanthan Gum |
| Flour | 6-10% | Oat Flour |
| Preservative Booster | 3% | Sodium Benzoate |
| Preservative Booster | 1.5% | Potassium Sorbate |
| Water Softener | 2.5% | Sodium Citrate |
| pH Adjuster/Acidic Cleaner | 3.5% | Citric Acid |
| Fragrance | 1-3% | |
| Liquid Surfactant | 0.5-2% | |
| Additives | 1-3% | |
| Drying Agent | 1-2% | |

### Example 2

| | % | Batch Weight |
|---|---|---|
| Ingredient | Weight | (pounds) |
| Galaxy Galsoft GLI 21 F | 47.25 | 23.6 |
| Sodium Hydroxypropyl Starch Phosphate | 13.78 | 6.9 |
| Seppic Solagum AX (Acacia and | 19.77 | 9.9 |
| Xanthan Gum) | | |
| Sodium Benzoate Powder | 2.36 | 1.2 |
| Potassium Sorbate Powder | 1.58 | 0.8 |
| Sodium Citrate | 2.36 | 1.2 |
| JBL Citrocoat N - Citric Acid | 3.94 | 2.0 |
| Charkit OAT COM (Colloidal Oatmeal) | 6.00 | 3.0 |
| Fragrance | 0.99 | 0.5 |
| Tocopheryl Acetate (Vitamin E) | 0.79 | 0.4 |
| Kaolin Clay | 1.18 | 0.6 |
| Total | 100.00 | 50.0 |

### Example 3

| | % | Batch Weight |
|---|---|---|
| Ingredient | Weight | (pounds) |
| Galaxy Galsoft GLI 21 F | 35.35 | 17.68 |
| Sodium Hydroxypropyl Starch Phosphate | 6.00 | 3.00 |
| Seppic Solagum AX (Acacia and | 30.20 | 15.10 |
| Xanthan Gum) | | |
| Sodium Benzoate Powder | 3.70 | 1.85 |
| Potassium Sorbate Powder | 2.50 | 1.25 |
| Sodium Citrate | 3.60 | 1.80 |
| JBL Citrocoat N - Citric Acid | 6.00 | 3.00 |
| Colloidal Oatmeal | 9.20 | 4.60 |
| Tocopheryl Acetate (Vitamin E) | 2.00 | 1.00 |
| Kaolin Clay | 1.45 | 0.73 |
| Total | 100.00 | 50.00 |

Galaxy Galsoft GLI 21 F in Examples 2 and 3 may be composed of disodium cocoyl glutamate (42-50% concentration), sodium cocoyl isethionate (18-24% concentration), sodium chloride (8-14% concentration), coco fatty acid (10-15% concentration), glutamic acid (5-10% concentration), water (4% maximum concentration).

The target weight of a sachet of the formulation in Example 2 is 68 grams, with a minimum weight of 65 grams and a maximum weight of 73 grams.

An exemplary bottle for use of the formulation in Example 2 may be 22 fluid ounces, which may allow for 18 fluid ounces for diluted cleanser and 4 fluid ounces of empty volume. The empty volume in the bottle may allow for better dissolution when the formulation is stirred or shaken in water. The amount of water to dissolve a sachet of the formulation in Example 2 to make 18 fluid ounces of diluted cleanser may be approximately 468 grams or 15.8 fluid ounces of water.

The formulation in Example 2 may be used to create a body wash.

The target weight of a sachet of the formulation in Example 3 is 20 grams, with a minimum weight of 17 grams and a maximum weight of 22 grams.

An exemplary bottle for use of the formulation in Example 3 may be 10 fluid ounces, which may allow for 8 fluid ounces for diluted cleanser and 2 fluid ounces of empty volume. The empty volume in the bottle may allow for better dissolution when the formulation is stirred or shaken in water. The amount of water to dissolve a sachet of the formulation in Example 3 to make 8 fluid ounces of diluted cleanser may be approximately 218 grams or 7.3 fluid ounces of water.

The formulation in Example 3 may be used to create a face wash.

1. A stable, anhydrous cleanser concentrate formulation, comprising an acidic cleaner, a pH control agent, one or more gelling agents, a drying agent, a water softener, and one or more surfactants.

2. The formulation of claim 1, further comprising one or more natural or synthetic fragrances.

3. The formulation of claim 1, further comprising a flour.

4. The formulation of claim 1, further comprising one or more preservative boosters.

5. The formulation of claim 1, wherein the one or more surfactants is 50-60% by weight of the formulation.

6. The formulation of claim 1, wherein the gelling agent is 10-15% by weight of the formulation.

7. The formulation of claim 1, wherein the acidic cleaner is 1-10% by weight of the formulation.

8. The formulation of claim 8, wherein the acidic cleaner comprises citric acid.

9. The formulation of claim 1, wherein the pH control agent is 1-10% by weight of the formulation.

10. The formulation of claim 9, wherein the pH control agent is citric acid.

11. The formulation of claim 1, wherein the drying agent is 1-5% by weight of the formulation.

12. The formulation of claim 2, wherein the one or more fragrances is 1-5% by weight of the formulation.

13. The formulation of claim 3, wherein the flour is 6-10% by weight of the formulation.

14. The formulation of claim 3, wherein the flour is oat flour.

15. The formulation of claim 4, wherein the one or more preservative boosters is 1-5% by weight of the formulation.

16. The formulation of claim 4, wherein the one or more preservative boosters comprises sodium benzoate and potassium sorbate.

17. The formulation of claim 1, wherein the formulation is a tablet.

18. The formulation of claim 1, wherein the formulation is a powder.

19. A method of use of the formulation of claim 1 wherein:
i) a container is filled with water;
ii) the formulation is added to the water forming a mixture; and
iii) the mixture is shaken or stirred for a period of time.

20. The method of claim 19, wherein the mixture is in a ratio of between 5:1 and 20:1 water to formulation.
